(19) United States
(12) Patent Application Publication
(10) Pub. No.: US 2007/0004652 A1
(43) Pub. Date: Jan. 4, 2007

(54) Treatment of occlusive thrombosis

(75) Inventors: Harold H. Schmitz, Bethesda, MD (US); Catherine L. Kwik-Uribe, Stroudsburg, PA (US); Paul G. Jones, Harby (GB)

Correspondence Address: NADA JAIN, P.C., 560 White Plains Road, Suite 460, Tarrytown, NY, 10591, US

(73) Assignee: Mars, Incorporated, McLean, VA (US)

(21) Appl. No.: 11/477,226

(22) Filed: Jun. 29, 2006

Related U.S. Application Data
(60) Provisional application No. 60/695,738, filed on Jun. 29, 2005.

(51) Int. Cl. A61K 31/7048; A61K 31/353

(52) U.S. Cl. 514/27; 514/456

(57) ABSTRACT

The invention relates to compositions, such as pharmaceuticals, foods, food additives, or dietary supplements, containing a flavanol, an A-type procyanidins, a B-type procyanidin or a derivative thereof, and methods of use thereof, for treatment and/or prevention of occlusive thrombosis and related conditions.

### FIELD OF THE INVENTION

The invention relates to compositions containing flavanols, A-type procyanidins, and/or B-type procyanidins and methods of use thereof, for prophylactic or therapeutic treatment of a human or a veterinary animal suffering from, or at risk of suffering from, an occlusive thrombus.

### BACKGROUND OF THE INVENTION

The normal process of the formation of the platelet plug (to prevent bleeding) may become pathological in the process of thrombosis in which a mass of platelets and fibrin forms within the arterial lumen.

The vast majority of arterial thrombotic episodes occur in arteries which have atherosclerosis. In atherosclerosis, lipid deposition leads to the formation of “plaques.” The initial step of plaque formation involves modification of plasma LDL which invokes monocyte adhesion to, and migration through, the intact endothelial surface. Within the intima, lipoproteins are further modified by oxidation and are taken by the monocytes to become lipid-filled foam cells to complete the first stage of atherosclerosis. This stage is manifested as a series of yellow dots or streaks visible to the naked eye on the intimal surface. Each fatty streak is a collection of lipid-filled foam cells within the intima. To this point, endothelial denudation has not occurred, and platelet adhesion plays no part in the initiation of plaques. The endothelial cells may overexpress adhesion molecules, have impaired nitric oxide (NO) synthesis or release, but there is no exposure of subendothelial collagen.

Plaque evolution to form an advanced lesion involves the recruitment of more macrophages and the formation of a core of extracellular lipid and cholesterol within the plaque. Concomitant with core formation, smooth muscle proliferation occurs, and these cells synthesize collagen to encapsulate the lipid. As further evolution of the plaque occurs, endothelial denudation occurs, and platelets are deposited. Thus, once a plaque has been initiated, platelet deposition becomes a factor in plaque growth. This ultramicroscopic thrombosis involves virtually all plaques beyond the fatty streak stage. Ultramicroscopic thrombi may have important pathophysiological implications but are far too small to obstruct flow. They are a marker of a dysfunctional endothelial surface in which control of vessel tone is abnormal and NO synthesis is impaired.

Two distinct mechanisms are responsible for the natural formation of larger thrombi over human coronary plaques. In the first, the endothelium is torn away and denudation is widespread. Thrombus forms over the plaque surface. This has been called superficial or level 1 plaque injury. In the second, a plaque tears open, exposing the depths of the lipid core to blood in the lumen. Blood enters the lipid core itself, coming into contact with fragments of collagen, crystals of cholesterol, and Tissue Factor produced by macrophages. This cocktail is a highly potent thrombogenic mixture, and thrombus forms within the plaque (deep or level 2 injury). Level 3 injury follows angioplasty, in which tears enter the media. This is not a natural cause of arterial thrombus. Both endothelial erosion and plaque rupture (level 1 and 2 injury) are usually complications of plaques with a high lipid component and extensive inflammation. The loss of endothelium leads to thrombi, which range from a millimeter across to occluding thrombi.

Occlusive thrombosis leading to myocardial infarction may develop very rapidly in a coronary artery or it may evolve over days. Sudden occlusive thrombosis usually indicates patients who have had major disruptions of a plaque, in which case the stimulus for thrombosis is very strong. A significant number of patients, have a powerful response to a small plaque event, suggesting that the systemic potential for thrombosis can be an important variable in determining individual outcome.

As the thrombus reaches the point of near or total occlusion, thrombus begins to propagate in the arterial lumen, usually downstream. This thrombus has different morphological characteristics, having a high content of red cells enmeshed in a matrix of fibrin. Myocardial infarction implies that complete occlusion has occurred for some hours. The structure of the final stage of occluding thrombus with a matrix of fibrin containing trapped red cells suggests it could easily be removed by fibrinolysis. Clinical studies confirm this view. For example, tPA (Tumor Plasminogen Activator) works by dissolving an occluding clot.

There remains a need in the art for treating occlusive thrombosis. A combination of in vitro and in vivo data obtained by Applicants support the concept that the compounds described herein may be used to provide a therapeutic option in the prevention of occlusive clot (thrombosis) formation (which can result in myocardial infarction, ischemic stroke, and DVT), dissolving the occlusive clot as well as serve as post-occlusive treatment following the occurrence of myocardial infarction, ischemic stroke, and DVT formation. By up-regulating the fibrinolytic system, the use of the compounds described herein may also reduce the risk of arterial and pulmonary embolus formation.

### SUMMARY OF THE INVENTION

The invention relates to compositions containing a flavanol, an A-type procyanidin, and/or a B-type procyanidin, and methods of use thereof, for prophylactic or therapeutic treatment of a human or a veterinary animal suffering from, or at risk of suffering from, occlusive thrombosis and conditions related thereto.

In one aspect, the invention relates to a composition, such as a pharmaceutical, a food, a food additive, or a dietary supplement comprising an effective amount of a flavanol, an A-type procyanidin and/or a B-type procyanidin. The composition may optionally contain an additional cardiovascular-protective or therapeutic agent, or may be administered in combination with such an agent. Also within the scope of the invention are packaged products containing the above-mentioned compositions and a label and/or instructions for use to treat or prevent occlusive thrombosis and related conditions.

In another aspect, the invention relates to methods of use of a flavanol, an A-type procyanidin, and/or a B-type procyanidin to treat or prevent occlusive thrombosis and related conditions.

### BRIEF DESCRIPTION OF THE DRAWINGS

FIGS. 1A-C represents B1 dimer-mediated changes in human umbilical vein endothelial cells (HUVEC) mRNA expression of tPA, uPA, and PAI. HUVEC were incubated with B! dimer at 5 μM for 0.5 and 24 hours, and the mRNA was isolated as detailed below in Example 1. TAQMAN assays were performed, and the results were expressed as relative abundance of mRNA expression for tPA (A), uPA 9(B), and PAI, respectively. Data are provided as means +/−SD and represent three independent experiments. The results of a statistical evaluation (T-test) are presented above each data column.

FIG. 2 represents B1 dimer-induced augmentation of tPA release from HUVEC. HUVEC were treated with B1 dimer at different concentrations for 24 hours, the medium was collected, and the tPA activity in the medium was measured. Data were expressed as tPA activity in units/ml [U/ml] and represent the mean +/−SD of n independent experiments (the value for n is provided above each treatment group). Statistical evaluations indicate that the B1 dimer mediated a dose-dependent increase in tPA release from HUVEC(* indicates significant difference from vehicle control).

FIG. 3 depicts treatments of HUVEC with B1 dimer that modulate the medium concentration of total PAI. HUVEC were treated with B1 dimer at different concentrations for 24 hours, the medium was collected, and the concentration of the total PAI (free and bound) was measured. Data were expressed as total PAI in ng/mL and represent the mean +/−SD of n independent experiments (the value for n is provided above each treatment group). Statistical evaluations indicate that the B1 dimer mediated a dose-dependent increase in tPA release from HUVEC (* indicates significant difference from vehicle control).

FIG. 4 depicts B1 ingestion that increases plasma tPA activity. The B1 dimer and vehicle were ingested by human volunteers applying a double-blind, cross-over design. Plasma tPA activity was assessed as detailed above, the data were normalized with regard to individual baselines, expressed as the mean tPA activity +/−SD (n=4) and plotted as a function of time. [*] Data points are statistically different as compared to the vehicle control at the same time.

FIG. 5 depicts B1 ingestion that increases plasma tPA activity. Each individual data set for plasma tPA activity was normalized with regard to baseline, plotted against time, and the individual AUCs [mU*ml⁻¹/240 min] were calculated. Data presented represent the mean +/−SD (n=4) of the individual AUCs for the ingestion of the B1 dimer or vehicle only, respectively.

FIG. 6 represents the TAQMAN® analysis of tPA expression in HUVECs.

FIG. 7 represents the TAQMAN® analysis of uPA expression in HUVECs.

FIG. 8 represents the TAQMAN® analysis of PAI 1 expression in HUVECs.

### DETAILED DESCRIPTION

All patents, patent applications and references cited in this application are hereby incorporated herein by reference. In case of any inconsistency, the present disclosure governs.

The invention relates to compositions comprising an effective amount of a flavanol, an A-type procyanidin and/or a B-type procyanidin, or a pharmaceutically acceptable salt or derivative thereof.

As used herein, the term “flavanol” or “flavan-3-ol” refers to a monomer and the term “procyanidin” refers to an oligomer.

The A-type procyanidin of the present invention is an oligomer composed of n monomeric, flavan-3-ol units of the formula: 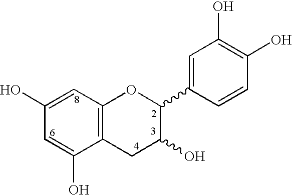
wherein
(i) the monomeric units are connected via interflavan linkages 4→6 and/or 4→8;
(ii) at least two of the monomeric units are additionally linked by an A-type interflavan linkage (4→8; 2→O→7) or (4→6; 2→O→7); and
(iii) n is 2 to 12.

It will be understood by a person of skill in the art that one of the two flavanol units linked by the A-type interflavanoid linkage must comprise two bonds at the 2- and 4-positions. Both of these have either α or β stereochemistry, i.e., the bonds are either 2α, 4α or 2β, 4β. These bonds connect to the 6- and 7-O-positions, or the 8- and 7-O-positions of the second flavanol unit linked by the A-type interflavan linkage. In constituent flavanol units of the oligomer which do not comprise A-type interflavan linkages at positions C-2 and C-4, the linkage at position C-4 can have either alpha or beta stereochemistry. The OH group at position C-3 of flavanol units has either alpha or beta stereochemistry. Flavan-3-ol (monomeric) units may be (+)-catechin, (−)-epicatechin and their respective epimers (e.g. (−)-catechin and (+)-epicatechin)).

An A-type procyanidin as defined above may be derivatized, for instance esterified, at one or more of the OH groups on one or more of the constituent flavan-3-ol units. A given flavan-3-ol unit may thus comprise one or more ester groups, preferably gallate ester groups, at one or more of the 3-, 5-, 7-, 3′- and 4′-ring positions. It may in particular be a mono-, di-, tri-, tetra- or penta-gallated unit.

Examples of the compounds useful for products, and in the methods of the present invention, include the compounds wherein the integer n is 3 to 12; 4 to 12; 5 to 12; 4 to 10; or 5 to 10. In some embodiments, n is 2 to 4, or 2 to 5, for example n is 2 or 3.

In one embodiment, the A-type procyanidin is epicatechin-(4β→8; 2β→O→7)-catechin (i.e., A1 dimer), or a pharmaceutically acceptable salt or derivative thereof, and has the following formula:

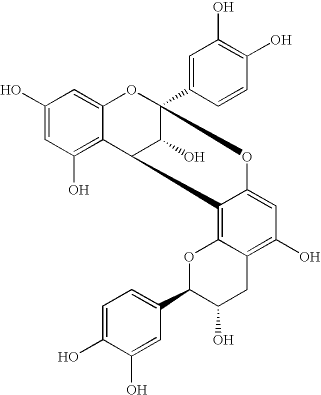

In another embodiment, the A-type procyanidin is epicatechin-(4β→8; 2β→O→7)-epicatechin (i.e., A2 dimer) and has the following formula:

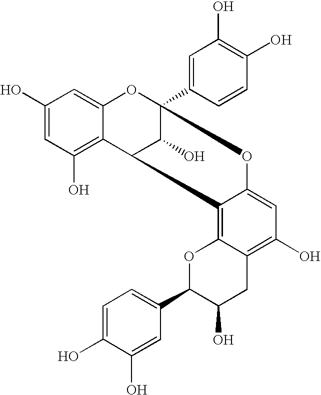

In yet another embodiment, the A-type procyanidin is an A-type trimer and has the following formula:

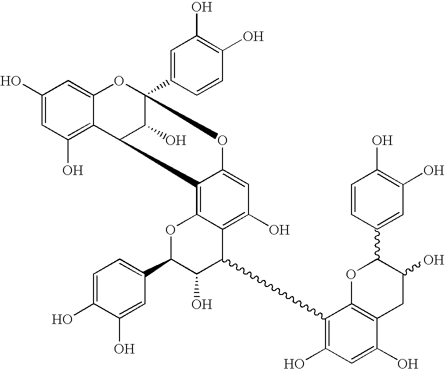

A-type procyanidins may be of natural origin or synthetically prepared. For example, A-type procyanidins may be isolated from peanut skins as described in Example 1, or as described in Lou et al., Phytochemistry, 51: 297-308 (1999), or Karchesy and Hemingway, J. Agric. Food Chem., 34:966-970 (1986), the relevant portions of each being hereby incorporated herein by reference. Mature red peanut skin contain about 17% by weight procyanidins, and among the dimeric procyanidins epicatechin-(4β→8; 2β→O→7)-catechin dominates, with smaller proportion of epicatechin-(4β→8; 2β→O→7)-epicatechin being present. However, in addition to procyanidins having (4→8; 2→O→7) double linkages, procyanidins having (4→6; 2→O→7) double linkages are also found in peanut skins.

Other sources of the above compounds are cranberries as described, for example in Foo et al., J. Nat. Prod., 63: 1225-1228, and in Prior et al., J. Agricultural Food Chem., 49(3):1270-76 (2001), the relevant portions of each being hereby incorporated herein by reference. Other sources include Ecdysanthera utilis (Lie-Chwen et al., J. Nat. Prod., 65:505-8 (2002)) and Aesculus hippocastanum (U.S. Pat. No. 4,863,956), the relevant portions of each being hereby incorporated herein by reference.

A-type compounds may also be obtained from B-type procyanidins via oxidation using 1,1-diphenyl-2-pycrylhydrazyl (DPPH) radicals under neutral conditions as described in Kondo et al., Tetrahedron Lett., 41: 485 (2000), the relevant portions of which are hereby incorporated herein by reference. Methods of obtaining natural and synthetic B-type procyanidins are well known in the art and are described, for example, in U.S. Pat. Nos. 6,670,390 to Romanczyk et al.; 6,207,842 to Romanczyk et al.; 6,420,572 to Romanczyk et al.; and 6,156,912 to Romanczyk et al, the disclosures of which are hereby incorporated herein by reference.

The A-type procyanidins may be used in the compositions described herein and administered in the form of an extract (e.g. peanut skins extract) comprising A-type procyanidins as the main component. The A-type procyanidins may be isolated and purified, i.e., they are separated from compounds with which they naturally occur (if the A-type procyanidin is of natural origin), or they are synthetically prepared, in either case such that the level of contaminating compounds (impurities) does not significantly contribute to, or detract from, the effectiveness of the A-type procyanidin. For example, an isolated and purified A1 dimer is separated from A2 dimer, with which it may occur in nature, to the extent achievable by the available commercially viable purification and separation techniques. The compounds may be substantially pure, i.e., they possess the highest degree of homogeneity achievable by the available purification, separation and/or synthesis technology. As used herein, a “substantially pure A1 dimer” is separated from A2 dimer to the extent technologically and commercially possible, and a “substantially pure A-type trimer” is separated from other A-type oligomers (to the extent permitted by the existing technology) but may contain a mixture of several A-type trimers. In other words, the phrase “isolated and purified trimer” refers primarily to one trimer, while a “substantially pure trimer” may encompass a mixture of trimers.

In some embodiments, the A-type procyanidins are at least 80% pure, preferably at least 85% pure, at least 90% pure, at least 95% pure, at least 98% pure, or at least 99% pure. Such compounds are particularly suitable for pharmaceutical applications.

The present invention also relates to a composition comprising an effective amount of the compound having the following formula Aₙ, or a pharmaceutically acceptable salt or derivative thereof (including oxidation products): 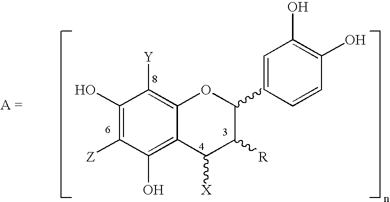
wherein
n is an integer from 2 to 18;
R and X each have either α or β stereochemistry;
R is OH, O-sugar or O-gallate;
the substituents of C-4, C-6 and C-8 are X, Z and Y, respectively, and bonding of monomeric units occurs at C-4, C-6 or C-8;
when any C-4, C-6 or C-8 are not bonded to another monomeric unit, each X, Y or Z is a hydrogen or a sugar; and
the sugar is optionally substituted with a phenolic moiety at any position, for instance, via an ester bond.

The sugar can be selected from the group consisting of glucose, galactose, rhamnose, xylose, and arabinose. The sugar is preferably a monosaccharide or di-saccharide. The phenolic moiety is selected from the group consisting of caffeic, cinnamic, coumaric, ferulic, gallic, hydroxybenzoic and sinapic acids. Monomeric units of the above formula Aₙ may be bonded via 4→6 and 4→8 linkages. Oligomers with exclusively (4→8) linkages are linear; while the presence of at least one (4→6) bond results in a branched oligomer. Also within the scope of the invention are oligomers comprising at least one non-natural linkage (6→6), (6→8), and (8→8).

Examples of the compounds of the formula Aₙ described herein are those having the integer n equal 2 to 18; 3 to 18; 2 to 12; 3 to 12; 2 to 5; 3 to 5; 4 to 12; 5 to 12; 4 to 10; or 5 to 10. Thus, B-type procyanidins within the scope of the above formula may be dimers, trimers, tetramers, pentamers, hexamers, heptamers, octamers, nonamers, and decamers, or mixtures of two or more of the aforementioned oligomers. In some embodiments n equals 2, i.e., the compound of formula Aₙ is a dimer.

In certain embodiments, the compound of the formula Aₙ is such that R is —OH, and/or X, Y, and Z are hydrogen. In other embodiments, the compound of formula Aₙ is such that R is —O-gallate and/or X, Y and Z are hydrogen. Examples of these compounds may be dimers, such as B₁, B₂ and B₅ dimers.

Thus, in one embodiment, the composition comprises an effective amount of the compound having the formula Aₙ, or a pharmaceutically acceptable salt or derivative thereof (including oxidation products): 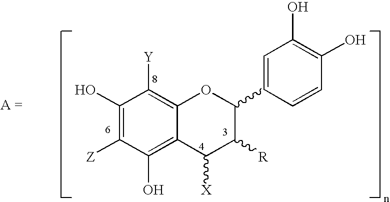
wherein
n is an integer from 2 to 18;
R and X each have either α or β stereochemistry;
R is OH;
the substituents of C-4, C-6 and C-8 are X, Z and Y, respectively, and bonding of monomeric units occurs at C-4, C-6 and C-8; and
when any C-4, C-6 or C-8 are not bonded to another monomeric unit, X, Y and Z are hydrogen.

The B-type procyanidins for use in the present invention may be of natural origin, for example, derived from a cocoa bean or another natural source of polyphenols, or prepared synthetically. For example, they may be prepared as described in U.S. Pat. Nos. 5,554,645; 6,670,390; 6,864,377; 6,420,572; 6,152,912; 6,476,241, the relevant portions of which are hereby incorporated herein by reference. A person of skill in the art may select natural or synthetic polyphenol based on availability or cost. Polyphenols may be included in the composition in the form of a cocoa ingredient containing cocoa polyphenols, for example, chocolate liquor included in chocolate, or may be added independently of cocoa ingredients, for example, as an extract, extract fraction, isolated and purified individual compound, pooled extract fractions or a synthetically prepared compound. The term “cocoa ingredient” refers to a cocoa solids-containing material derived from shell-free cocoa nibs such as chocolate liquor and partially or fully-defatted cocoa solids (e.g. cake or powder).

Also within the scope of the invention are flavanols and compositions comprising an effective amount of a flavanol. Examples of flavanols are epicatechin and catechin, such as (−)-epicatechin and (+)-catechin.

Flavanol and/or procyanidin derivatives may also be useful. These include esters of monomer and oligomers such as the gallate esters (e.g. epicatechin gallate and catechin gallate); compounds derivatized with a saccharide moiety such as mono- or di-saccharide moiety (e.g. β-D-glucose), metabolites of the procyanidin monomers and oligomers, such as the glucuronidated and methylated derivatives, and oxidation products. Oxidation products may be prepared as disclosed in U.S. Pat. No. 5,554,645, the relevant portions of which are incorporated herein by reference. Esters, for example esters with gallic acid, may be prepared using known esterification reactions, and for example as described in U.S. Pat. No. 6,420,572, the disclosure of which is hereby incorporated herein by reference. Methylated derivatives, such as 3′O-methyl-, 4′O-methyl-, and 3′O, 4′O-dimethyl-derivatives may be prepared, for example, as described in Cren-Olive et al., 2002, J. Chem. Soc. Perkin Trans. 1, 821-830, and Donovan et al., Journal of Chromatography B, 726 (1999) 277-283, the disclosures of which are hereby incorporated herein by reference. Glucuronidated products may be prepared as described in Yu et al, “A novel and effective procedure for the preparation of glucuronides.” Organic Letters, 2(16) (2000) 2539-41, and as in Spencer et al, “Contrasting influences of glucuronidation and O-methylation of epicatechin on hydrogen peroxide-induced cell death in neurons and fibroblasts.” Free Radical Biology and Medicine 31(9) (2001) 1139-46.

Methods of Use

Any compound and composition described in the application may be used to practice the methods described herein.

Methods of treating and/or preventing occlusive thrombosis (i.e., treatment and/or prevention of stable clots) by administering to a human or a veterinary animal suffering from, or at risk of, suffering from occlusive thrombosis are within the scope of the invention. Genetic factors such as Factor V Leiden can indicate an increased risk of occlusive thrombosis. As discussed in the Background, occlusive clots may result in myocardial infarction, ischemic stroke or DVT, and arterial or pulmonary embolism.

Thus, the compounds and compositions described herein may be administered to subjects that are diagnosed with a developing occlusive clot to break down the clot, and/or to prevent or reduce the risk of myocardial infarction, ischemic stroke or DVT, and arterial or pulmonary embolism. The compounds may also be administered for post-occlusive clot formation and/or post event therapy, i.e., after the occurrence of myocardial infarction, ischemic stroke or DVT, and/or arterial or pulmonary embolism. Subjects suffering from a vascular event/incident have a greater risk of suffering from another, thus the compounds of the invention may be administered protectively as a post-event therapy.

The term “preventing” means reducing the risks associated with developing a disease and/or a condition, including reducing the onset of the disease and/or the condition. For example, genetic factors such as Factor V Leiden can indicate an increased risk of occlusive thrombosis.

The effective amount for use in the above methods may be determined by a person of skill in the art using the guidance provided herein and general knowledge in the art. For example, the effective amount may be such as to achieve a physiologically relevant concentration in the body (e.g. blood) of a mammal. Such a physiologically relevant concentration may be at least about 10 nanomolar (nM), preferably at least about 20 nM, or at least about 100 nM, and more preferably at least about 500 nM. In one embodiment, at least about one micromole in the blood of the mammal, such as a human, is achieved. The compounds of formula Aₙ, as defined herein, may be administered at from about 50 mg/day to about 1000 mg/day, preferably from about 100-150 mg/day to about 900 mg/day, and most preferably from about 300 mg/day to about 500 mg/day. However, amounts higher than stated above may be used. The amounts may be determined as described in Adamson, G. E. et al., J. Ag. Food Chem.; 1999; 47 (10) 4184-4188, the disclosure of which is hereby incorporated herein by reference.

The compounds may be administered acutely, or treatments/preventive administration may be continued as a regimen, i.e., for an effective period of time, e.g., daily, monthly, bimonthly, biannually, annually, or in some other regimen, as determined by the skilled medical practitioner for such time as is necessary. The administration may be continued for at least a period of time required to exhibit therapeutic/prophylactic effects. Preferably, the composition is administered daily, most preferably two or three times a day, for example, morning and evening to maintain the levels of the effective compounds in the body of the mammal. To obtain the most beneficial results, the composition may be administered for at least about 30, or at least about 60 days. These regiments may be repeated periodically.

Compositions and Formulations

The compounds of the invention may be administered as a pharmaceutical, food, food additive or a dietary supplement.

As used herein a “food” is a material containing protein, carbohydrate and/or fat, which is used in the body of an organism to sustain growth, repair and vital processes and to furnish energy. Foods may also contain supplementary substances such as minerals, vitamins and condiments. See Merriam-Webster's Collegiate Dictionary, 10th Edition, 1993. The term food includes a beverage adapted for human or animal consumption. As used herein a “food additive” is as defined by the FDA in 21 C.F.R. 170.3(e)(1) and includes direct and indirect additives. As used herein, a “pharmaceutical” is a medicinal drug. See Merriam-Webster's Collegiate Dictionary, 10th Edition, 1993. A pharmaceutical may also be referred to as a medicament. As used herein, a “dietary supplement” is a product (other than tobacco) that is intended to supplement the diet that bears or contains the one or more of the following dietary ingredients: a vitamin, a mineral, an herb or other botanical, an amino acid, a dietary substance for use by man to supplement the diet by increasing the total daily intake, or a concentrate, metabolite, constituent, extract or combination of these ingredients.

Pharmaceuticals containing the inventive compounds, optionally in combination with another cardiovascular-protective or therapeutic agent, may be administered in a variety of ways such as orally, sublingually, bucally, nasally, rectally, intravenously, parenterally and topically. A person of skill in the art will be able to determine a suitable mode of administration to maximize the delivery of a flavanol, A-type procyanidin, and/or B-type procyanidin, optionally in combination with another cardiovascular-protective or therapeutic agent. Thus, dosage forms adapted for each type of administration are within the scope of the invention and include solid, liquid and semi-solid dosage forms, such as tablets, capsules, gelatin capsules (gelcaps), bulk or unit dose powders or granules, emulsions, suspensions, pastes, creams, gels, foams, jellies or injection dosage forms. Sustained-release dosage forms are also within the scope of the invention. Suitable pharmaceutically acceptable carriers, diluents, or excipients are generally known in the art and can be determined readily by a person skilled in the art. The tablet, for example, may comprise an effective amount of a flavanol, A-type procyanidin, and/or B-type procyanidin containing composition and optionally a carrier, such as sorbitol, lactose, cellulose, or dicalcium phosphate. A person of skill in the art can determine the most suitable mode of administration, e.g. I.V. (being the fastest way to deliver a compound, I.V. administration can be used where mediation of an immediate effect is needed), oral administration (may be chosen for subsequent event prevention).

The dietary supplement containing a flavanol, A-type procyanidin, and/or a B-type procyanidin, or pharmaceutically acceptable salts or derivative thereof, and optionally another cardiovascular-protective or therapeutic agent, may be prepared using methods known in the art and may comprise, for example, ingredients such as dicalcium phosphate, magnesium stearate, calcium nitrate, vitamins, and minerals.

As used herein, the terms “cardiovascular-protective or therapeutic agent” refers to an agent other than flavanol, A-type procyanidin or B-type procyanidin which is effective to treat or protect cardiovascular system. Examples of such agents are anti-platelet therapy agents (e.g. COX inhibitors, such as aspirin); NO-modulating agents; cholesterol reducing agents (e.g. sterol, stanol); and anti-coagulant/blood-thinning agents (e.g. herparin, warfarin).

Further within the scope of the invention is an article of manufacture such as a packaged product comprising the composition of the invention (e.g. a food, a dietary supplement, a pharmaceutical) and a label indicating the presence of, or an enhanced content of the inventive compounds or directing use of the composition for methods described herein.

Also within the scope of the invention is an article of manufacture (such as a packaged product or kit) adapted for use in combination therapy comprising at least one container and at least one flavanol, A-type procyanidin, and/or B-type procyanidin, or a pharmaceutically acceptable salt or derivatives thereof. The article of manufacture further comprises at least one additional agent, a cardiovascular-protective or therapeutic agent (i.e., other than the flavanol, A-type procyanidin, B-type procyanidin, or a pharmaceutically acceptable salt or derivative thereof), which agent may be provided as a separate composition, in a separate container, or in admixture with the compound of the invention.

The foods comprising flavanols, A-type and/or B-type procyanidins and/or their derivatives, and optionally another cardiovascular-protective/treatment agent, may be adapted for human or veterinary use, and include pet foods. The food may be other than a confectionery, however, the preferred cholesterol lowering food is a confectionery such as a standard of identity (SOI) and non-SOI chocolate, such as milk, sweet and semi-sweet chocolate including dark chocolate, low fat chocolate and a candy which may be a chocolate covered candy. Other examples include a baked product (e.g. brownie, baked snack, cookie, biscuit) a condiment, a granola bar, a toffee chew, a meal replacement bar, a spread, a syrup, a powder beverage mix, a cocoa or a chocolate flavored beverage, a pudding, a rice cake, a rice mix, a savory sauce and the like. If desired, the foods may be chocolate or cocoa flavored. Food products may be chocolates and candy bars, such as granola bars, containing nuts, for example, peanuts, walnuts, almonds, and hazelnuts. In one embodiment, the nut skins, e.g. peanut skins, are added to the nougat of a chocolate candy.

A daily effective amount of flavanols and/or A-type and/or B-type procyanidins may be provided in a single serving. Thus, a confectionery (e.g. chocolate) may contain at least about 100 mg/serving (e.g. 150-200, 200-400 mg/serving).

The invention is further described in the following non-limiting examples.

### EXAMPLES

### Example 1

### Extraction and Isolation of A-type Procyanidins

Extraction

Finely ground peanut skins (498 g) were defatted with hexane (2×2000 mL). Hexane was removed by centrifugation at ambient temperature, 5 min at 3500 rpm, and discarded. Residual hexane was allowed to evaporate overnight. The following day, defatted peanut skins were extracted for 2 hours at ambient temperature with acetone:water:acetic acid (70:29.5:0.5 v/v/v) (2×2000 mL). Extracts were recovered by centrifugation (ambient temperature, 5 min at 3500 rpm). Organic solvents were removed by rotary evaporation under partial pressure (40° C.). Aqueous portion of extraction solvent was removed by freeze drying to provide a brown-red crusty solid (51.36 g).

Gel Permeation of Crude Peanut Skin Extract

Crude peanut skin extract (24 g), obtained as described above, was dissolved in 70% methanol (150 mL), refrigerated for 1 hour, vortexed for 3 sec, then centrifuged at ambient temperature, for 5 min at 3500 rpm. The supernatant was loaded atop a large column containing Sephadex LH-20 (400 g) preswollen in methanol. Column was eluted isocratically with 100% methanol at a flow rate of 10 mL/min. Twenty nine fractions, 250 mL each, were collected and combined in accordance to their composition as determined by NP-HPLC (Adamson et al., J. Ag. Food Chem., 47: 4184-4188, 1999) to give a total of eight fractions (i-viii). Fraction i contained monomers epicatechin and catechin, fraction ii-vii contained dimers, trimers or mixtures thereof. Fraction v (1.8 g) and vii (2.7 g) contained a preponderance of dimers and trimers, respectively, and were selected for further purification.

Purification of A-type Dimers and Trimers

Fraction v (1.8 g) was dissolved in 0.1% acetic acid in 20% methanol (40 mg/mL). Injection volumes were 2 mL. Separations were conducted on a Hypersil ODS (250×23 mm) under gradient conditions. Mobile phases consisted of 0.1% acetic acid in water (mobile phase A) and 0.1% acetic acid in methanol (mobile phase B). Gradient conditions were: 0-10 min, 20% B isocratic; 10-60 min, 20-40% B linear; 60-65 min, 40-100% B linear. Separations were monitored at 280 nm. Fractions with equal retention times from several preparative separations were combined, rotary evaporated at 40° C. under partial vacuum and freeze dried. Five fractions (a-e) were obtained. Fractions d and e were characterized by LCMS as dimers A1 and A2, respectively. In addition to A1 and A2 dimers, four different dimers were previously isolated from peanut skins (Lou et al., Phytochemistry 51, 297-308, 1999).

Fraction vii was purified as described above to obtain a single trimer with an A-linkage having the formula represented above.

The structures of purified compounds were confirmed by Mass Spectroscopy, and the purity of the compounds was determined using HPLC at UV 280 nm. A1 dimer was 95% pure, A2 dimer was 91% pure, and A trimer was 84% pure.

### Example 2

The following experiments show that (−)-epicatechin (including a mixture of (−)-epicatechin metabolites), procyanidin dimer B1, and procyanidin dimer A2 can have pronounced effects on the expression and secretion of proteins integral to controlling stable clot (thrombus) formation, specifically tissue plasminogen activator (tPA), urokinase-type plasminogen activator (uPA), and plasminogen activator inhibitor 1 (PAI-1).

We have employed a genomic approach to comprehensively investigate the effects of (−)-epicatechin (including a mixture of (−)-epicatechin metabolites), procyanidin dimer B1, and procyanidin dimer A2 on the gene expression of human endothelial cells in vitro. Following the completion of an extensive evaluation utilizing an Affymetrix Oligonucleotide Microarray Gene Expression Analysis System, we have subsequently validated our findings using Taqman® Gene Expression Assays, and confirmed the ensuing data by directly assessing amounts or activities of the target proteins in cultured human endothelial cells and human plasma, respectively.

Taken together, our results demonstrate that (−)-epicatechin (including a mixture of (−)-epicatechin metabolites), the procyanidin dimer B1, and the procyanidin dimer A2 modulated the expression, secreting or activity of various proteins related to cardiovascular function. The information provided below will focus on one group of such proteins that is closely related to the regulation of thrombosis and fibrinolysis, and thus closely associated with cardiovascular health and disease, namely tPA (tissue plasminogen activator), uPA (urokinase, or urinary plasminogen activator) and PAI (plasminogen activator inhibitor).

The B1 Dimer Modulates the Gene Expression of tPA and PAI from Human Umbilical Vein Endothelial Cells In Vitro

Methodological Background

Human umbilical vein endothelial cells (HUVEC) were cultured in an endothelium-specific, 2% serum-containing, growth factor-supplemented, antibiotic-free culture medium. Cryo-preserved cells from a single, male, Caucasian donor in passage 1 or 2 were directly seeded into fibronectin-coated 6 well plates at a seeding density of 5000 cells/cm² and cultured without sub-culturing using standard cell culture conditions. 50% of the medium was replaced with fresh medium every 24 h until confluence. Cells were treated with the B1 dimer at a final concentration of μ5 M for 0.5, 2, 4, and 24 hours, respectively, and mRNA was isolated with a Qiagen mRNA Isolations System. cDNA was synthesized from mRNA samples using a HPLC-purified T7 Oligo(dT) primer and SuperScript II reverse transcriptase enzyme (Invitrogen). The ensuing cDNA samples were purified using a Qiagen PCR purification system. The cDNA templates were added to standardized Taqmang® Gene Expression Assays (Applied Biosystems) reactions mixtures and a Real-Time PCR was performed using standardized thermo-cycling conditions. An absolute quantification method of analyzing gene expression levels was used on triplicate reactions of each sample, and amplification plots generated by the Applied Biosystems 7900HT Fast Real-Time PCR System were analyzed using ABI Prism SDS v2.1 software.

B1 Dimer-Mediated Changes in tPA, uPA, and PAI mRNA Expression

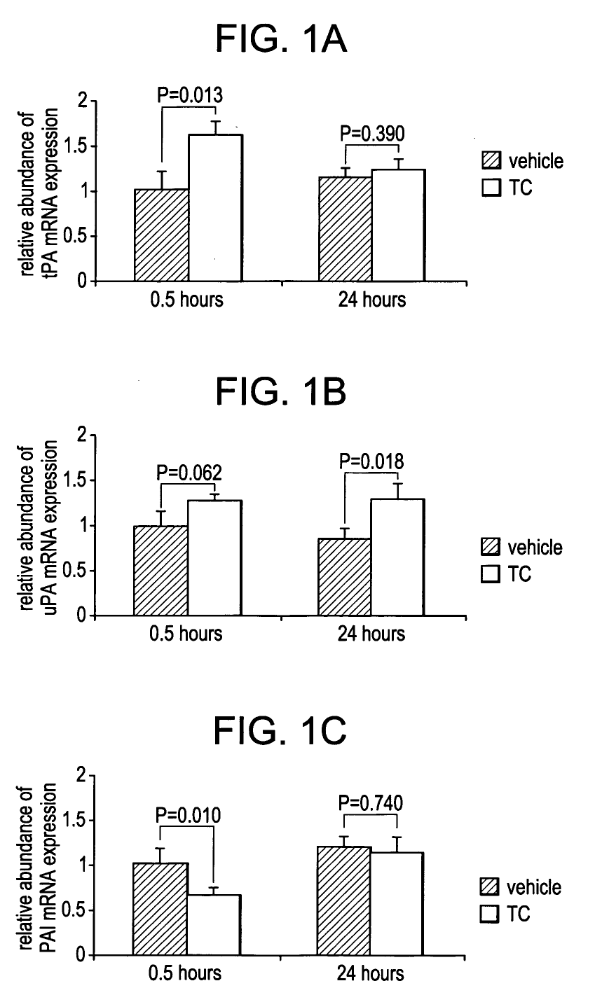

FIG. 1 demonstrates that the administration of the B1 dimer to HUVEC cultures mediated time-dependent increases in the MRNA expression for tPA (FIG. 1A), uPA (FIG. 1B), and decreases the expression of PAI mRNA (FIG. 1C).

The B1 Dimer Modulates the Release of tPA from Human Umbilical Vein Cells In Vitro

Methodological Background

HUVEC cultures were established as detailed above, by seeding HUVEC into fibronectin-coated 6 well plates at a seeding density of 5000 cells/cm². Cells were cultured without sub-culturing using standard cell culture conditions. 50% of the medium was replaced with fresh medium every 24 h. Following an incubation of the HUVECs with the TC at a concentration of 5 μM for 24 h, an aliquote of the media was collected and analyzed for its content of tPA, and PAI, respectively. The remaining medium was collected and the weight was recorded for subsequent activity calculations (for calculation purposes we assumed that 1 g of medium=1 mL). tPA and PAI release was measured as the activity of the respective proteins in the collected media using an ELISA-based assay [Innovative Research Inc., Southfield, Mich., USA] in accordance with the manufacturer's instructions.

The B1 Dimer Mediates an Increase in tPA Activity in HUVEC Culture Medium

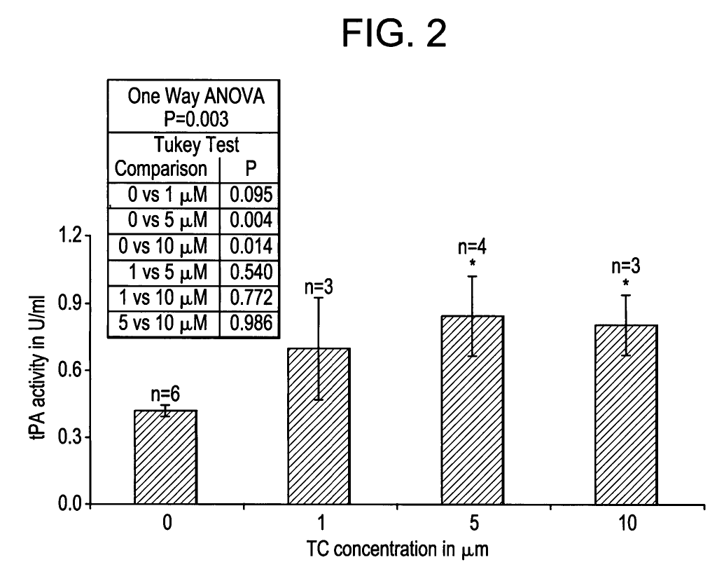

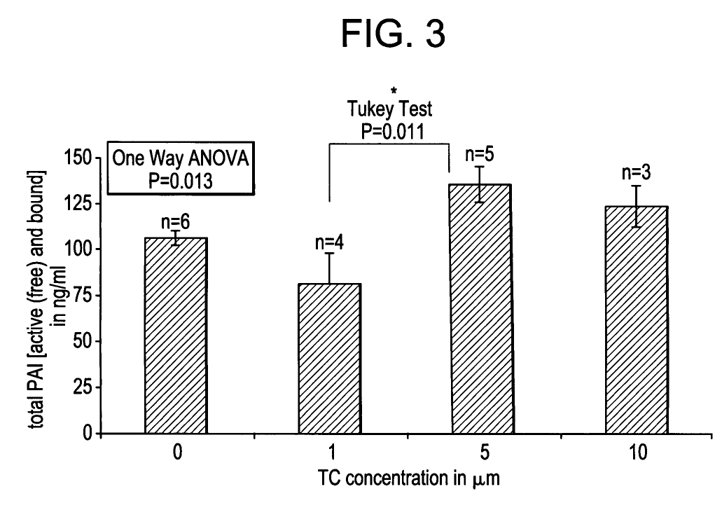

As demonstrated in FIG. 2, treatments of HUVEC with the B1 dimer resulted in an increase in tPA activity in the cell culture medium following 24 hours of incubation. The effect of the B1 dimer on HUVEC tPA release is dose-dependant (FIG. 2) and the increases in media-present tPA activity at B1 dimer concentrations of 5 μM and 10 μM are significantly different from vehicle treatments, respectively (One Way ANOVA followed by Tukey Test, FIG. 2). Measurements of the activity of PAI in the media seemingly indicated that the B1 dimer caused a dose-dependant decrease in PAI activity. However, these changes were not quite statistically significant (P=0.061, One Way ANOVA). Increasing the current number of independent experiments (n) may be advised in order to obtain a higher statistical power. In addition, measurements of total PAI (free and tPA/uPA-bound), demonstrated that the TC exerted a significant dose-dependent effect, causing an increase in total PAI at a concentration of 5 μM as compared to 1 μM (FIG. 3, One Way ANOVA followed by Tukey Test).

Human B1 Dimer Ingestion and the Acute Modification of Plasma tPA, uPA, and PAI Levels

Methodological Background

The test compound was administered to human volunteers in accordance with IRB-approved protocols and as detailed in the previous section of this report. The activity of tPA, uPA, and PAI in plasma was measured using an ELISA-based assay [Innovative Research Inc., Southfield, Mich., USA] in accordance with the manufacturer's instructions.

tPA

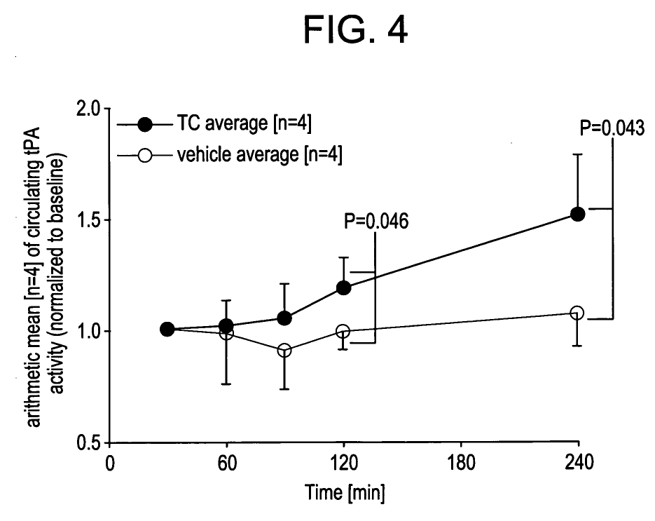

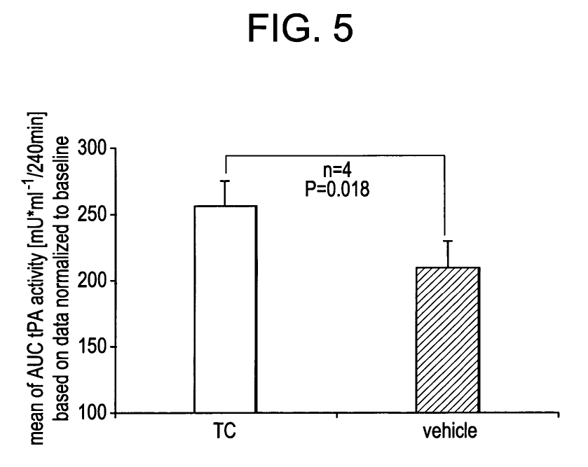

Resultant from the non-transformed (raw) data set, the ingestion of the B1 dimer caused a time-dependent increase in plasma tPA activity [One Way ANOVA, P=0.333], whereas the ingestion of vehicle alone did not have an effect [P=0.803]. Based on the mean (n=4) maximal increases in plasma tPA activity [tPAₘₐₓ] it can be demonstrated that the ingestion of the B1 dimer caused a 46% increase in tPAₘₐₓ [mean tPAₘₐₓ=261.4+/−39.4 mU/mL], whereas the ingestion of the vehicle alone mediated a tPAₘₐₓ of 16%. For the purpose of further comparisons, the data have been normalized with regard to the individual baseline values [FIG. 4]. The results also show that the ingestion of the B1 dimer resulted in an augmentation of the closure time as compared to the ingestion of the vehicle (water) [FIG. 4]. In order to remove variations that are based on individual differences with regard to the time-dependency of the effect, the individual area under the curves (AUC), based on the normalized data set [FIG. 4], were calculated and are presented in FIG. 5. As can be ascertained from the data provided [FIG. 4], the plasma tPA activity does not return to baseline values during the time course of observation, thus we would suggest to extend the time course and to increase the number of volunteers, should further investigations be conducted.

In addition to measurements of plasma tPA activity, we determined the amount of tPAₜₒₜₐₗ in plasma (tPAₜₒₜₐₗ=free and bound tPA). Based on the non-transformed (raw) data set, neither the ingestion of the B1 dimer nor that of the vehicle caused a time-dependent change in plasma tPAₜₒₜₐₗ levels [One Way ANOVA, P=0.769 and P=0.812, respectively]. The arithmetical average of all measurements indicates that the plasma concentration for tPAₜₒₜₐₗ equals 6.8+/−1.6 ng/mL.

uPA and PAI

Resultant from the non-transformed (raw) data set, the ingestion of the B1 dimer did not cause a statistically significant, time-dependant change in plasma uPA and PAI activities. However, this may be based on the fact that one volunteer showed PAI values that were at baseline already 400% higher than those of the other three volunteers. Based on the normalization of the data set with regard to the individual baselines, the ingestion of the B1 dimer, but not that of the vehicle control, time-dependently decreased the PAI plasma activity with statistical significance at 4 h post-ingestion {P=0.011, t-test).

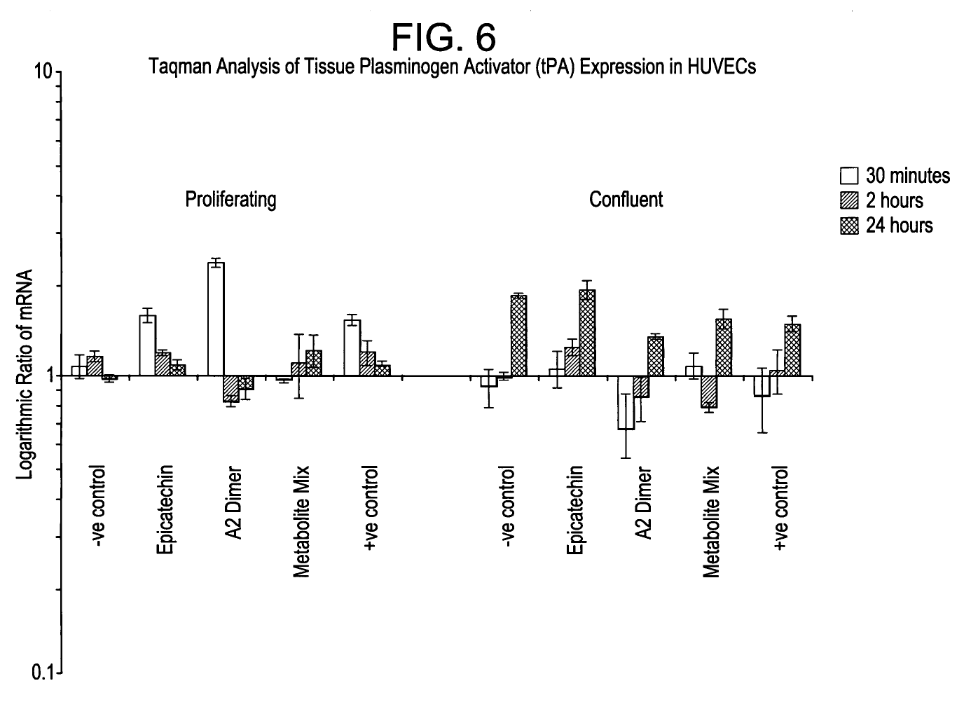

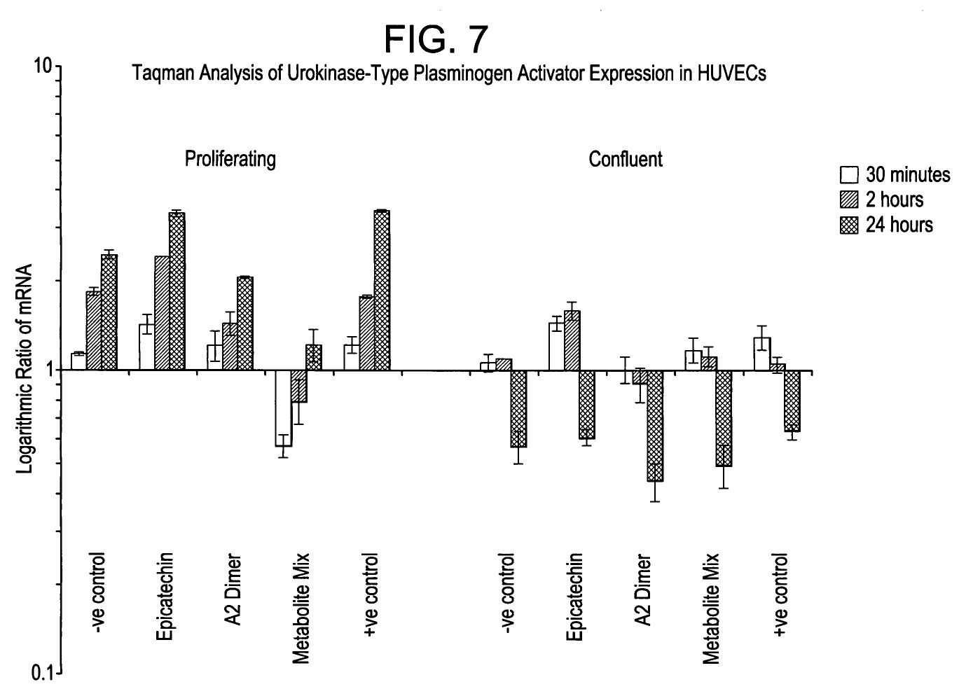

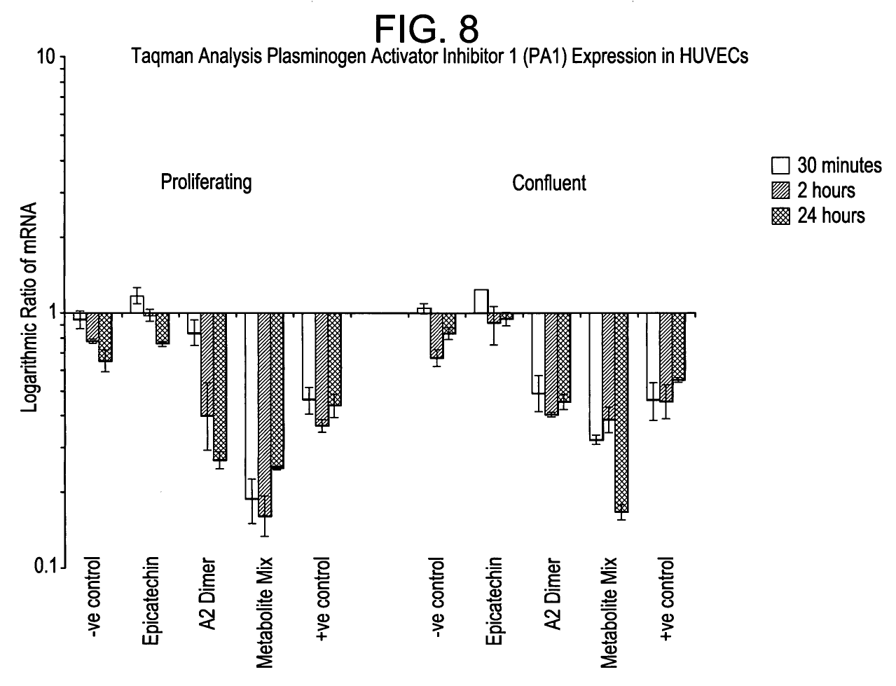

FIGS. 6, 7, and 8 show the data with (−)-epicatechin (including a mixture of (−)-epicatechin metabolites), procyanidin dimer B1, and procyanidin dimer A2, and their effect on tPA, uPA, or PAI expression in HUVECs.

What is claimed is:

1. A method of treating or preventing occlusive thrombosis by administering to a subject in need thereof an effective amount of an A-type procyanidin composed of n monomeric units of the formula: 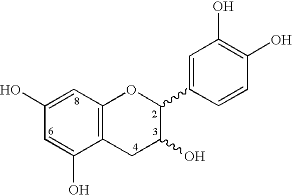
wherein
(i) the monomeric units are connected via interflavan linkages 4→6 and/or 4→8;
(ii) at least two of the monomeric units are additionally linked by an A-type interflavan linkage (4→8; 2→O→7) or (4→6; 2→O→7);
(iii) n is 2 to 12;
or a pharmaceutically acceptable salt or derivative thereof,
and wherein the subject is a human or a veterinary animal.

2. The method of claim 1, wherein the A-type procyanidin is isolated and purified.

3. The method of claim 1, wherein the A-type procyanidin is a dimer.

4. The method of claim 3, wherein the dimer is A2 dimer.

5. The method of claim 4, wherein A2 dimer is isolated and purified.

6. The method of claim 1, wherein the subject is a human suffering from an occlusive thrombus.

7. The method of claim 1, wherein the subject is a human at risk of myocardial infarction, ischemic stroke, DVT, or arterial or pulmonary embolism.

8. A method of treating or preventing occlusive thrombosis by administering to a subject in need thereof an effective amount of the compound having the following formula Aₙ, or a pharmaceutically acceptable salt or derivative thereof (including oxidation products): 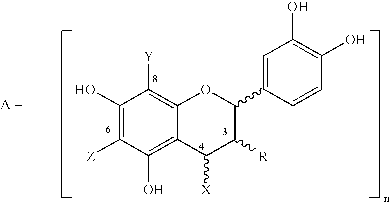
wherein
n is an integer from 2 to 18;
R and X each have either α or β stereochemistry;
R is OH, O-sugar or O-gallate;
the substituents of C-4, C-6 and C-8 are X, Z and Y, respectively, and bonding of monomeric units occurs at C-4, C-6 or C-8;
when any C-4, C-6 or C-8 are not bonded to another monomeric unit, each X, Y or Z is a hydrogen or a sugar; and
the sugar is optionally substituted with a phenolic moiety at any position, for instance, via an ester bond.

9. The method of claim 8, wherein R is —OH and X, Y and Z are hydrogen.

10. The method of claim 8, wherein n is 2.

11. The method of claim 9, wherein n is 2.

12. The method of claim 8, wherein the compound is a B1 dimer.

13. The method of claim 8, wherein the subject is a human suffering from an occlusive thrombus.

14. The method of claim 8, wherein the subject is a human at risk of myocardial infarction, ischemic stroke, DVT, or arterial or pulmonary embolism.

15. A method of treating or preventing occlusive thrombosis by administering to a subject in need thereof an effective amount of a compound selected from the group of a flavanol and a derivative thereof.

16. The method of claim 15, wherein the compound is epicatechin.

17. The method of claim 16, wherein epicatechin is (−)-epicatechin.

18. The method of claim 15, wherein the derivative is a methylated derivative.

19. The method of claim 15, wherein the subject is a human suffering from an occlusive thrombus.

20. The method of claim 15, wherein the subject is a human at risk of myocardial infarction, ischemic stroke, DVT, or arterial or pulmonary embolism.

21. The method of claim 17, wherein the subject is a human suffering from an occlusive thrombus.

22. The method of claim 17, wherein the subject is a human at risk of myocardial infarction, ischemic stroke, DVT, or arterial or pulmonary embolism.
